(19) United States
(12) Patent Application Publication
(10) Pub. No.: US 2011/0245856 A1
(43) Pub. Date: Oct. 6, 2011

(54) POROUS ACUPUNCTURE NEEDLE

(75) Inventor: Kou-Tsair SU, Kaohsiung (TW)

(73) Assignees: TAIWAN SHAN YIN INTERNATIONAL CO., LTD.; BIOMATE MEDICAL DEVICES TECHNOLOGY CO., LTD.

(21) Appl. No.: 12/749,959

(22) Filed: Mar. 30, 2010

(51) Int. Cl. A61B 17/34

(52) U.S. Cl. 606/189

(57) ABSTRACT

A porous acupuncture needle includes a needle portion for puncturing the outer skin of human body, and an operation portion for being held and handled by a user. The needle portion includes a needle body, a porous layer formed by a plurality of micropores on the surface of the needle body, and a charge layer covering on the surface of the porous layer. When performing acupuncture, the affinity provided by the charge layer makes the human tissues to attach to the needle portion more easily. Moreover, during twirling the needle, the porous layer also increases the capability to affect the surrounding tissues and therefore the stimulation to the acupuncture point.

### FIELD OF THE INVENTION

The present invention is related to an acupuncture needle, and more particularly to a porous acupuncture needle.

### BACKGROUND OF THE INVENTION

Acupuncture is one kind of therapies usually used in traditional Chinese medicine. This therapy is especially effective in curing chronic diseases and easing pains, so that much attention is paid thereto. During acupuncture therapy, the doctor usually uses a needle to insert into the acupuncture point on human body, and the needle is so called the acupuncture needle. In the early stage, the acupuncture needle is formed by stone, rock, bone or bamboo, and it is gradually developed to use metal to form the needle. Among others, the needle made of stainless steel is featured of hard, flexible, tough, antirust, heat-resistant and inexpensive, so that it is widely used nowadays.

Generally, the structure of acupuncture needle includes a needle stem at one end, a needle point at the other end to puncture the human skin and a needle portion at the center with flexibility. Currently, the surface of the acupuncture needle formed by industrial fabrication method is very smooth, so that when the doctor processes the puncture, due to the flexibility of human skin and the inner pressure of human body, the patient might feel uncomfortable at the moment the needle punctures through the skin. The prior arts, such as U.S. Pat. No. 3,976,078 and China Patent No. 1247178, had developed a needle with slots formed on the needle point and the needle portion for reducing the pain during the puncture. However, as the doctor further executes the manipulations of the needle such as lifting and thrusting the needle, or twirling the needle, the needle with the smooth surface or a small number of unidirectional slots might provide a limited capability to affect the surrounding tissues, and the acupuncture point may not be stimulated effectively. Besides, as the needle is inserted into the body tissue, the metal surface of the conventional acupuncture needle lacks of affinity to the surrounding tissues and also insufficient for bio-compatibility, the therapeutic effect of the acupuncture might be influenced.

### SUMMARY OF THE INVENTION

The object of the present invention is to increase the capability of the acupuncture needle to stimulate the acupuncture point on human body.

For achieving the object described above, the present invention provides a porous acupuncture needle including a needle portion for puncturing the outer skin of human body, and an operation portion for being held and handled by a user. The needle portion includes a needle body, a porous layer formed by a plurality of micropores on the surface of the needle body, and a charge layer covering on the surface of the porous layer. The charge layer is a negative charge layer covering the surface of the porous layer, particularly the charge layer may be a hydroxyl layer. In addition, the micropores can be communicated with each other for forming the porous layer to be a three-dimensional porous structure.

In the present invention, the micropores respectively have a diameter ranged from 20 to 1000 nm, and the porous layer has a thickness ranged from 50 to 5000 nm. And, the porous layer has a dielectric constant ranged from 2 to 110 F/M.

According to the porous acupuncture needle of the present invention, the porous layer on the needle portion can effectively reduce the resistance at the moment of puncture for releasing the pain and discomfort of the patient, and also can improve the capability to affect the surrounding tissues as lifting and thrusting the needle or twirling the needle. Besides, the charge layer also provides a physical potential difference to effectively increase the stimulation capability to the acupuncture point. Furthermore, the porous layer can improve the affinity of the acupuncture needle to the human tissues, so that the human tissues can attach to the acupuncture needle more effective.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIG. 1A is a sectional view showing the structure of a porous acupuncture needle in a preferred embodiment according to the present invention;

FIG. 1B is a partial magnification view showing a porous layer in FIG. 1A;

FIG. 2 is an electron microscope photograph showing the surface of a porous acupuncture needle in a preferred embodiment according to the present invention; and

FIG. 3 is a graph showing a result of methylene blue test of a porous acupuncture needle in a preferred embodiment according to the present invention.

### DESCRIPTION OF THE PREFERRED EMBODIMENT

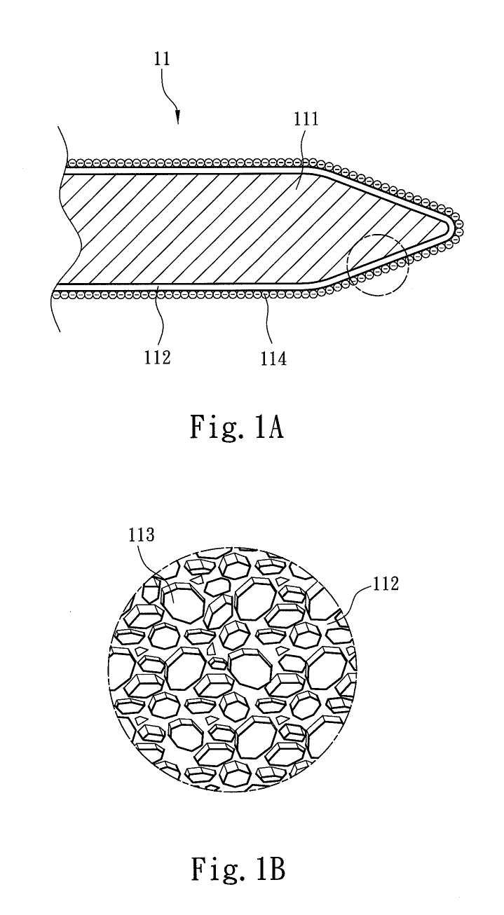

Please refer to FIG. 1A and FIG. 1B which are respectively a sectional view and a partial magnification view showing the structure of a preferred embodiment according to the present invention. As shown, the present invention is a porous acupuncture needle including a needle portion 11 for puncturing the outer skin of human body, and an operation portion (not shown in the drawings) for being held and handled by the user. The needle portion 11 includes a needle body 111, a porous layer 112 formed by a plurality of micropores 113 on the surface of the needle body 111, and a charge layer 114 covering on the surface of the porous layer 112.

In this embodiment, the charge layer 114 is a negative charge layer which may be made of hydroxyl groups (OH⁻). The porous layer 112 has a dielectric constant ranged from 2 to 110 F/M. The needle portion 11 can be made of metal material, including iron (Fe), titanium (Ti), cobalt (Co), nickel (Ni), chromium (Cr), aluminum (Al), vanadium (V), or an alloy thereof. The micropores 113 in the porous layer 112 on the needle portion 11 respectively have a diameter ranged from 20 to 1000 nm, and the porous layer 112 has a thickness ranged from 50 to 5000 nm. The micropores 113 can be individually formed on the porous layer 112 or can be communicated with each other for forming the porous layer 112 to be a three-dimensional porous structure, so that through the three-dimensional porous structure, the surface area of the acupuncture needle can be increased to carry more charges. In addition, the communicated micropores 113 allow air flowing therebetween, so that during puncture, the inner pressure of human body can be released from the micropores 113, thereby reducing the patient's discomfort.

In the present invention, the charge layer 114 can be formed by photocatalytic reaction process or chemical process. In the photocatalytic reaction process, the needle portion 11 is illuminated by an excitation light source for a proper period of time. The excitation light can be ultraviolet (UV), laser, plasma, or halogen light. When the needle portion 11 receives the illumination of the excitation light source, the electrons on the surface thereof absorb a sufficient amount of energy for escaping the energy level, so as to form positive electric holes. Then, as the positive electric holes contact with other surrounding molecules, such as water or hydroxyl groups, the hydroxyl radicals are generated to form the charge layer 114, so as to provide the acupuncture needle with oxidization ability. In the chemical process, the acupuncture needle can be soaked in a hydrogen peroxide solution for a proper period of time, so as to perform oxidation on the surface of the needle portion 11, thereby forming the charge layer 114 of hydroxyl groups.

### Example I

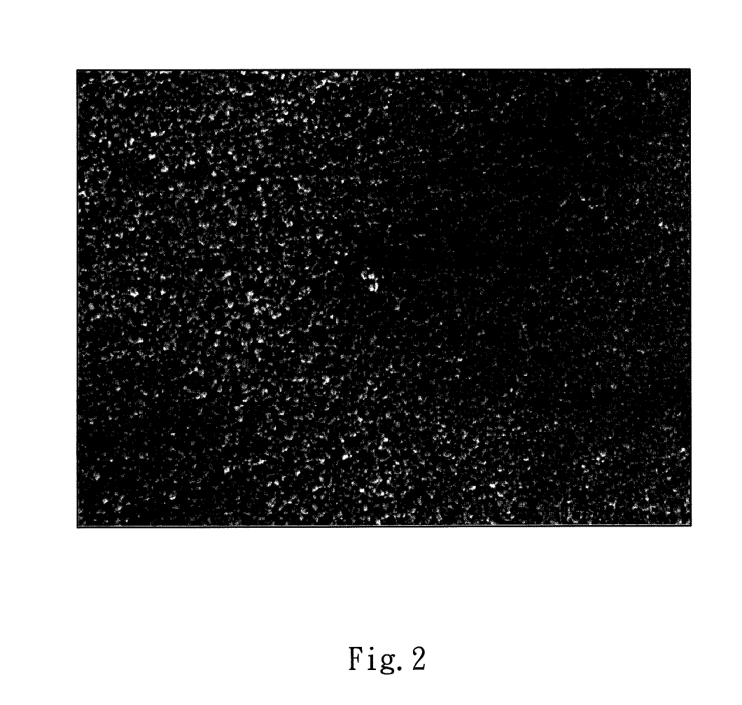

In this embodiment, an acupuncture needle is processed by 10% hydrogen peroxide solution for 10 hours to obtain the porous acupuncture needle according to the present invention. The processed porous acupuncture needle is observed under the electron microscope (SEM, JEOL JSM-6500). During observation, an acceleration voltage of 15 kV is applied to a vacuum environment (9.63*10⁻⁵ Pa). As shown in FIG. 2, the micropores in the porous layer on the porous acupuncture needle of the present invention can have a diameter in nanoscale after processing.

### Example II

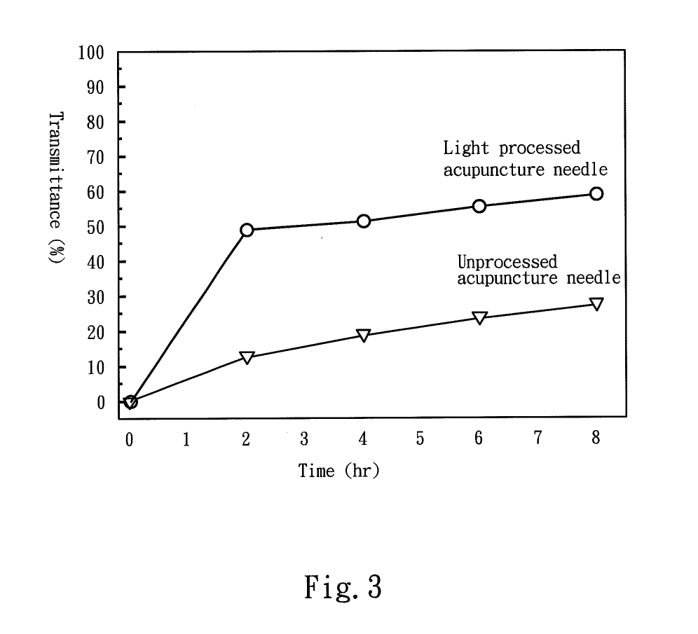

In this embodiment, the porous acupuncture needle according to the present invention carries out the methylene blue test. After processed by UV for 24 hours, the obtained porous acupuncture needle and an unprocessed acupuncture needle are respectively placed in a 10 ppm methylene blue solution (10 ml). After reacted in a dark environment for 30 minutes, the respective solutions are tested by Agilent 8453 UV-vision Spectrophotometer, and recorded the transmittance of methylene blue solutions every 2 hours. The test result is shown in FIG. 3.

According to the test result shown in FIG. 3, it can be found that the methylene blue solution of the processed acupuncture needle has a better transmittance than that of the unprocessed acupuncture needle. It means the processed acupuncture needle has photocatalytic effect.

In the aforesaid, by means of placing the charge layer on the surface of the needle portion, the affinity of the acupuncture needle to the human tissues surrounded can be increased and the bio-compatibility thereof also can be improved. Also, the human tissues can be attached to the needle portion easily. Moreover, the charge layer also provides a surface potential difference for the needle portion, which can increase the stimulation to the tissues around the acupuncture point. Furthermore, the porous layer formed with a plurality of micropores can significantly increase the surface area of the acupuncture needle. Compared with the conventional acupuncture needles with smooth surface or with unidirectional slots mounted thereon, the charged amount of the charge layer can become higher. Besides, at the moment of puncture, the porous layer can effectively reduce the resistance for releasing the pain and discomfort of the patient. As lifting and thrusting the needle or twirling the needle, the capability to affect the surrounding tissues can also be improved.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A porous acupuncture needle, comprising a needle portion for puncturing the outer skin of human body, and an operation portion for being held and handled by a user, characterized in that:
the needle portion comprises a needle body, a porous layer formed by a plurality of micropores on the surface of the needle body, and a charge layer covering on the surface of the porous layer.

2. A porous acupuncture needle as claimed in claim 1, wherein the micropores are communicated with each other for forming the porous layer to be a three-dimensional porous structure.

3. A porous acupuncture needle as claimed in claim 1, wherein the charge layer is a negative charge layer.

4. A porous acupuncture needle as claimed in claim 3, wherein the charge layer is a negative charge layer of hydroxyl groups.

5. A porous acupuncture needle as claimed in claim 1, wherein the micropores respectively have the diameter ranged from 20 to 1000 nm.

6. A porous acupuncture needle as claimed in claim 1, wherein the porous layer has a thickness ranged from 50 to 5000 nm.

7. A porous acupuncture needle as claimed in claim 1, wherein the porous layer has dielectric constant ranged from 2 to 110 F/M.
